(19) United States
(12) Patent Application Publication
(10) Pub. No.: US 2022/0177251 A1
(43) Pub. Date: Jun. 9, 2022

(54) AN AUTOMATIC DISPENSER

(71) Applicant: JARYD RIDGEWAY, Johannesburg (ZA)

(72) Inventor: JARYD RIDGEWAY, Johannesburg (ZA)

(21) Appl. No.: 17/441,584

(22) PCT Filed: Mar. 18, 2020
(86) PCT No.: PCT/ZA2020/050016

§ 371 Date: Sep. 21, 2021

(30) Foreign Application Priority Data
Mar. 26, 2019 (ZA) 2019/01841

(51) Int. Cl. B65H 29/12; A61B 42/40; B65H 5/28; B65H 16/00; B65H 16/10

(52) U.S. Cl. CPC B65H 29/12; A61B 42/40; B65H 5/28; B65H 2515/40; B65H 16/10; B65H 2403/92; B65H 16/005

(57) ABSTRACT

A dispenser, apparatus, system and method for automatically dispensing portable or sterile items from a continuous length of packages of items ideally packaged as a stack, including a support shaped and configured to support the stack, a feed mechanism arranged in flow communication relative to the support for feeding the stack towards a dispensing point, a separation means extending from the feed mechanism, shaped and configured for separating an item from the stack at the dispensing point, an electric motor configured to drive the feed mechanism; and a sensor shaped and configured to detect a dispensing parameter, wherein in use, the dispenser is activated by a user providing the dispensing parameter to the sensor thereby activating the feed mechanism to automatically feed the stack towards the dispensing point such that the separation means separates the item from stack thereby allowing for the item to be dispensed.

### FIELD OF THE INVENTION

The present invention relates to a dispenser for dispensing a sterile item, more particular dispenser for automatically dispensing a single sterile item from a continuous length of individually-packaged items.

### BACKGROUND TO THE INVENTION

Conventional dispensers are well known and available in the market place. Examples include automated teller machines (ATMs) for dispensing cash, food vending machines for dispensing food and toilet paper dispensers for dispensing toilet paper.

There are several disadvantages associated with dispensers in the market, for example: dispensing of irregularly-shaped objects either requires vibration, specific item orientation arrangements before loading into the dispenser or a specific mechanism to stop any jamming during operation of the dispenser.

There are several technological advances related to dispensers in the market place, more specifically in the automation technology area thereby creating a niche automatic dispenser market. The automation of dispensers allows dispensers to either dispense singular and/or multiple items which is desirous in high volume dispensing environments, for example in medical and laboratory environments.

It shall be appreciated that workspace in general commands a premium in medical and laboratory environments therefore, a compact dispenser is desirous for such environments as opposed to bulky dispensers common in the market place.

The challenge however faced by medical and laboratory environments is one of hygiene, in that it is desirous to dispense items in a sterile manner, for example items such as gloves, surgical equipment, needles etc. These items are typically sterile, therefore they undergo sterilisation in accordance with the products requirements, such as UV exposure, Ethylene Oxide (EtO), auto-cleaving and or other procedures beforehand before being, typically, packaged in a sterile manner. Items are typically packaged in a singular form, for example each item is packed in some form of packaging.

It shall be appreciated that the term “hygiene” may be construed to refer to but not limited to conditions or practices conducive to maintaining health and preventing disease, and/or contamination especially through cleanliness and/or avoidance of a transmission of pathogens and/or microbes.

It is an object of the invention to provide for compact, cost effective, efficient automatic dispenser for dispensing sterile items.

### SUMMARY OF THE INVENTION

Wherever used in this document:

“stack” means a continuous length of packages of items, this length rolled into and stored as a stack; and

“item” means any portable item such as a glove or a pair thereof, surgical equipment, needles, syringe or any other item practically applicable for use with the invention.

According to a first aspect of the invention, there is provided an automatic dispenser for dispensing sterile items from a stack, which dispenser includes a housing having a roller carrier arranged thereon, shaped and configured to rotationally support the stack, a feed mechanism for feeding the stack towards a dispensing point, which feed mechanism is arranged in flow communication relative to the roller carrier, a separation means extending from the feed mechanism, which separation means is shaped and configured for separating an item from the stack at the dispensing point, an electric motor configured to drive the feed mechanism; and a sensor arranged relative to the housing, which sensor is shaped and configured to detect a dispensing parameter, wherein in use, the dispenser is activated by a user providing the dispensing parameter to the sensor thereby activating the feed mechanism to automatically feed the stack towards the dispensing point such that the separation means separates the item from stack thereby allowing for the item to be dispensed.

The automatic dispenser may include a power source configured for powering the dispenser. The power source may be configured to provide electrical power in the form of conventional direct current (DC), for example as rechargeable batteries or in the form of alternating current (AC).

The feed mechanism may include two spaced apart wheel and cog arrangements.

The feed mechanism may include a third cog or a geared shaft interconnected to the motor, which third cog or geared shaft is shaped and configured to co-axially engage with the spaced apart wheel and cog arrangements.

The separation means may include a pair of spaced apart spindles, which spindles extend from the spaced apart wheel and cog arrangement.

Each package may include two layers of plastics material, plastics like material or any other flexible material joined to define a cavity in which at least an individual item is locatable, the layers being joined to define lines or regions of weakness defining a transverse locus and a longitudinal locus.

The dispenser may include a programmable computer processor arranged in electrical communication with the power source and the feed mechanism, which processor is configured to process drive the feed mechanism in a determinable manner as pre-programmed, in use.

The sensor may be any one or a combination of the following: an electrical, electro-mechanical, mechanical or electronic arrangement capable of detecting an electrical, electro-mechanical, mechanical or electronic parameter and being triggered by the dispensing parameter from a first state in which the arrangement does not activate the feed mechanism, to a second state in which the arrangement activate the feed mechanism.

According to a second aspect of the invention, there is provided a method for dispensing sterile items from a stack, which method includes the steps, of:
a) loading the stack onto an automatic dispenser in accordance with this invention,
b) securing layers of the packaging of the stack onto the separation means;
c) actuating an electrical motor to thereby drive a feed mechanism for feeding the stack towards a dispensing point;
d) feeding the stack via the feed mechanism towards the dispensing point located proximal to the separation means; and
e) separating an item from the stack using the separation means which separation means is shaped and configured to separate the item from the stack.

Alternatively, the step (b) may precede the step (a).

According to a third aspect of the invention, there is provided an automatic dispensing apparatus for dispensing an item from a continuous length of packages of items, including a support shaped and configured to support the length of packaged items, a feed mechanism for feeding the length of packages of items towards a dispensing point, a separation means adapted to separate layers of packaging of the length of packages of items at the dispensing point, an actuator configured to drive the feed mechanism and the separation means; and a sensor in communication with the actuator adapted to detect a dispensing parameter and to thereby output an initiating signal, wherein the apparatus is activated upon the actuator receiving the initiating signal, thereby driving the feed mechanism to automatically feed the length of packages of items towards the dispensing point and the separation means to separate the layers of packaging at a portion of the length of packages of items proximal to the dispensing point, thereby allowing for at least the item to be dispensed.

The packaging may include two layers of plastics material, plastics like material or any other flexible material joined such that they define a cavity in which at least the item is locatable.

The support may be a roller carrier adapted to rotationally support the length of packages of items as a stack.

The apparatus may include a power source configured for providing operational power to the apparatus in the form of direct current (DC) or alternating current (AC).

The feed mechanism may include a first cog and a second cog distanced apart and rotatable substantially in unison and substantially at the same velocity.

The feed mechanism may include a third cog interconnected and driven by the actuator, the third cog engaged with the first and second cogs to transfer driving force from the actuator to the first and second cogs, thereby rotating co-axially therewith.

The separation means may include a pair of spaced apart spindles, coupled to or attachable to the first and second cogs to extend therefrom to respectively rotate in unison with the cogs.

Each spindle may have a surface adapted to be attachable to a respective layer of packaging, or may be integrally attached to the respective layer of packaging.

The apparatus may include a programmable computing processor arranged in electrical communication with the sensor, the power source and the actuator, configured to control functional parameters of the actuator in a determinable manner, in use. One such parameter may be speed of rotation supplied to the cogs.

Preferably, the sensor is a motion sensor or a heat sensor, with the dispensing parameter respectively being movement within a predetermined distance of the sensor or heat of a predetermined magnitude.

The actuator may be a motor.

According to a fourth aspect of the invention there is provided an automatic dispensing system including a continuous length of packages of items and an automatic dispensing apparatus as described above, wherein the continuous length of packages of items is provided on a support of the automatic dispensing apparatus.

The system may be operated according to the method of the second aspect of the invention.

### BRIEF DESCRIPTION OF THE DRAWINGS

The invention is now described, by way of non-limiting example, with reference to the accompanying drawings wherein:

FIG. 1 shows a preferred embodiment of an automatic dispenser and dispensing apparatus according to a first aspect and a third aspect of the invention, in an inoperative condition;

FIG. 2 is an exploded view of the dispenser of FIG. 1;

FIG. 3 is an alternate isometric view of the dispenser of FIG. 1;

FIG. 4 is a sectional side view of an automatic dispensing system according to a fourth aspect of the invention;

FIG. 5 is a front view of the dispensing system of FIG. 4 in direction F;

FIG. 5A is a cross-sectional view of a section A of the system of FIG. 5; and

FIG. 6 is a view of the dispenser of FIG. 2 with front covers 12a and 12b omitted.

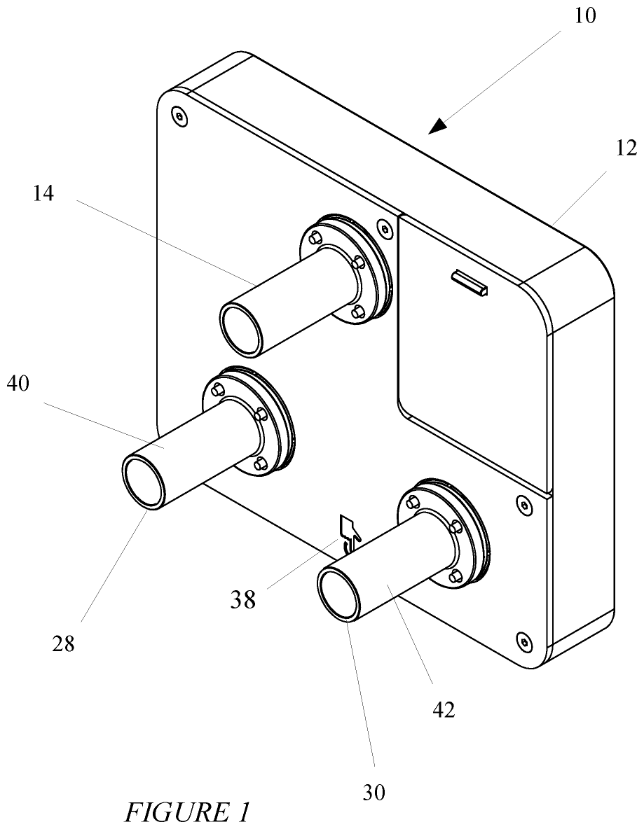

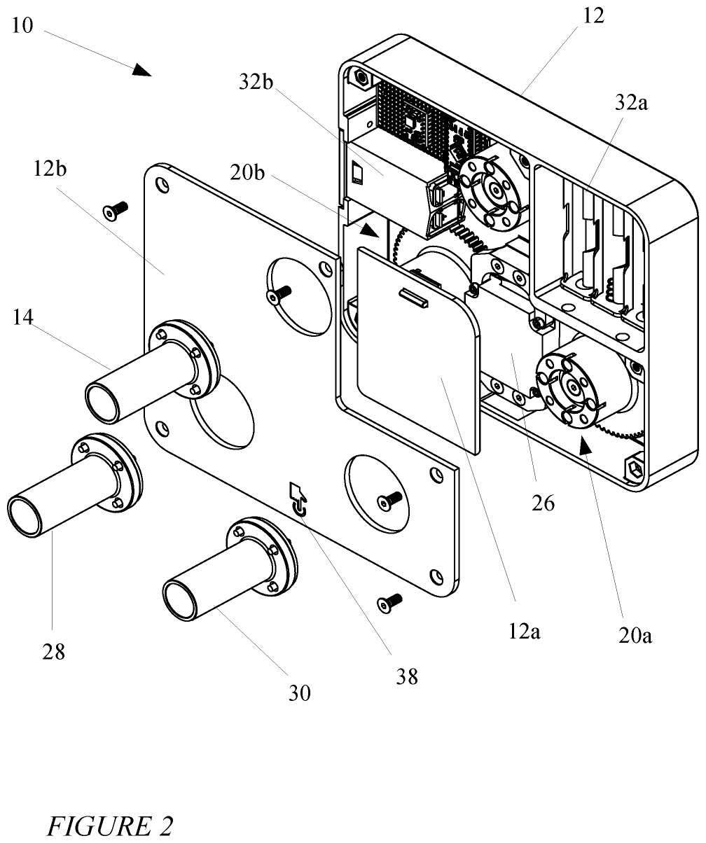

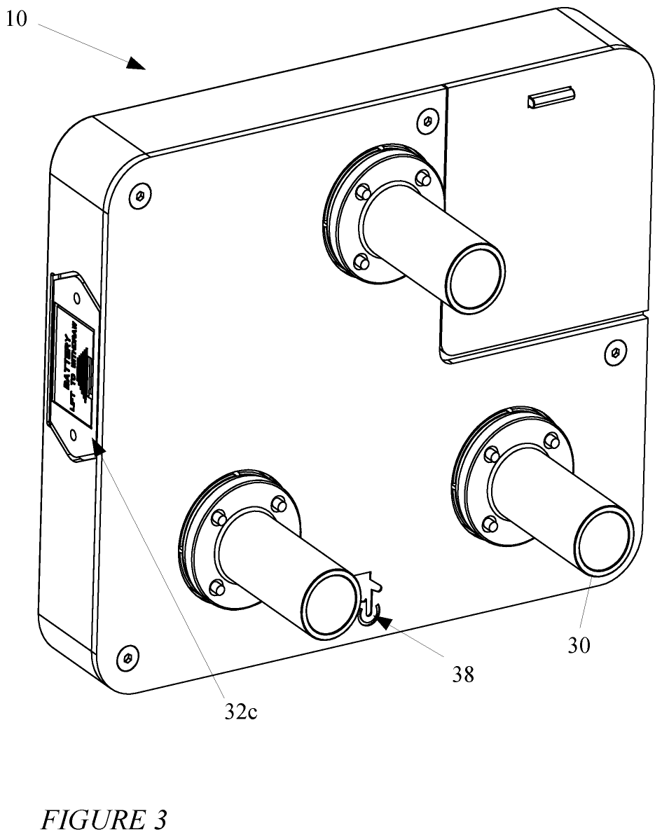

### DESCRIPTION OF PREFERRED EMBODIMENTS

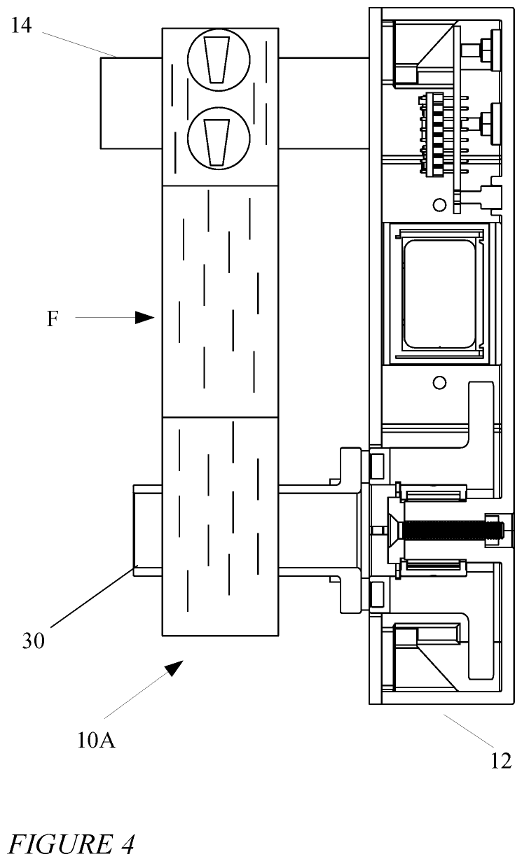

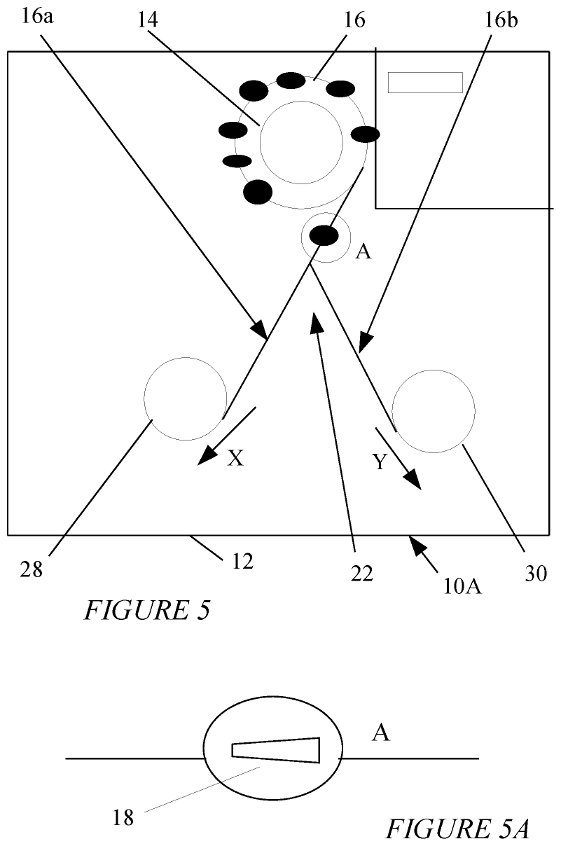

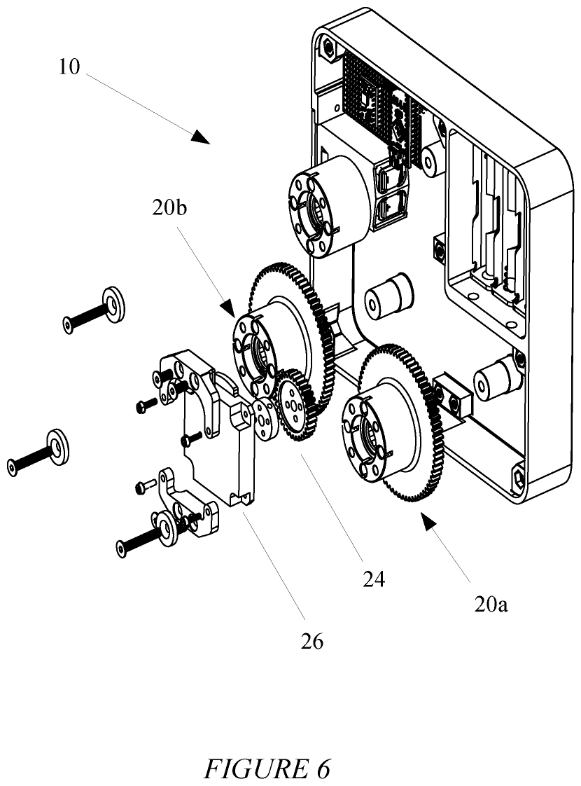

With reference to the accompanying drawings, an automatic dispenser in accordance with a first aspect of the invention is indicated generally by reference numeral 10. The dispenser 10 is used in an automatic dispensing system 10A in accordance with a fourth aspect of the invention as shown in FIGS. 4 and 5.

The automatic dispenser 10 includes a housing 12 having a roller carrier 14 arranged thereon, shaped and configured to rotationally support a stack 16 of individually-packaged items, specifically in this embodiment, the items are sterile individually packaged micro-centrifuge tubes 18. The dispenser 10 further includes a feed mechanism in the form of spaced apart mobile two spaced apart wheel and cog arrangements 20a and 20b configured for feeding the stack 16 towards a dispensing point 22.

The wheel and cog arrangements 20a and 20b are arranged in flow communication relative to the roller carrier 14. A third cog 24 interconnected to an electrical motor 26, which third cog 24 is shaped and configured to co-axially engage with the spaced apart wheel and cog arrangements 20a and 20b, such that in use, the third cog 24 coaxially engages with wheel and cog arrangement 20a and 20b.

A separation means in the form of spaced apart spindles 28 and 30 is arranged to extend from free ends of each wheel and cog arrangement 20a and 20b. The spaced apart spindles 28 and 30 have tabs 40 and 42 arranged which tabs 40 and 42 are configured for separating items from the packaging o the stack 16.

A power source 32a in the form of conventional direct current (DC) rechargeable batteries are used to power the dispenser 10. Ideally a battery level indicator 32b is present in communication with the power source 32a. An alternative power source port 32b provides connectivity of the dispenser 10 to an alternating current (AC) electrical supply. This port 32b, when connected to the AC supply, is configured to function as the power source 32a.

The dispenser 10 further includes a programmable computer processor 36 arranged in electrical communication with the power source 32a and the wheel and cog arrangements 20a and 20b, which processor 36 is configured to process input information for optimally driving the third cog 24 that in turn coaxially drives the wheel and cog arrangements 20a and 20b in a determinable manner as pre-programmed, in use. Such a determinable manner is, ideally, rotation substantially unison and substantially at the same velocity, and length of time of such rotation.

A motion sensor 38 arranged on the housing 12, the sensor shaped and configured to detect a dispensing parameter, namely a user's input for dispensing an item. The processor 36 is configured to process input information for optimally driving the third cog 24 that in turn coaxially drives the wheel and cog arrangements 20a and 20b such that the stack 16 is urged towards the dispensing point 22. At this dispensing point 22, the spaced apart spindles 28 and 30 are configured to separate the micro-centrifuge tube 18 from stack 16.

With regard to the system 10A:

The packaging essentially comprises a dual layer of packaging, a first layer 16a of a plastic like material and a second layer 16b of manufactured plastic like material with the micro-centrifuge tube 18 sandwiched in between the first and second layers 16a and 16b with the edges of the packaging sealed hermetically. Lines or regions of weakness (not referenced) are defined on the packaging between the layers 16b and 16b where the layers 16a and 16b are joined, defining a transverse locus and a longitudinal locus, each locus configured for a tearing action along said the lines or regions of weakness, to expose the micro-centrifuge tube 18 and to facilitate for a sterile, automatic and sequential dispensing of individual micro-centrifuge tubes 18 from the stack 16.

In use therefore, the stack 16 is mounted on the roller 14 of the dispenser 10. The stack 16 is urged is manually at its free end towards the spaced apart spindles 28 and 30 at the dispensing point 22. The packaging is then arranged on the tabs 40 and 42 in the following manner: The first layer 16a is attached to the first tab 40 and the second layer 16b is also attached to the second tab 42 with the micro-centrifuge tube 18 sandwiched in between the first and second layers 16 a and 16b of the packaging.

The dispenser 10 is then activated by the user who supplies the dispensing parameter, in this case the dispensing parameter being motion, to the sensor 38 to thereby trigger input to the computer processor 36. The processor 36 is arranged in electrical communication with the power source 32 and the motor 26, completing the circuitry required for the power source 32a to provide power to the motor 26. The motor then drives the third cog 24, which cog 24 then drives the wheel and cog arrangements 20a and 20b. The stack 16 is thereby urged towards the dispensing point 22 such that the first layer 16a is collected at the first tab 40, urged in direction X with a concomitant urging of the second tab 42 in direction Y and with the layer 16b collected at tab 42. As shown, directions X and Y are diametrically opposed directions. This results in a tearing action along the lines of weakness along the transverse locus and the longitudinal locus as the packaging is unwrapped thus dispensing the micro-centrifuge tube 18.

The processor 36 has a pre-set rotation period of the motor 26 and stops the motor 16 after the dispensation of the micro-centrifuge tube 18. It is further envisaged that improved efficiency may be gained in alternative embodiments of the invention by locating a radio-frequency identification (“RFID”) tag (not illustrated) on the packaging of the stack 16. Such tag would be preloaded with information relating to optimal dispensing of the micro-centrifuge tubes 18. The information may include the time of turning and the velocity of the motor 26 required to dispense optimally each tube micro-centrifuge 18 without exposing the next micro-centrifuge tube 18 on the packaging. The sensor 38 would be pre-configured to synchronise with the tag upon the tag coming within predetermined proximity to the sensor 38.

In order to dispense of the next micro-centrifuge tube 18 on the stack 16, the user henceforth merely provides a successive dispensing parameter. The spindles 28 and 30, under the driving force of the motor 26, then rotate to automatically dispense the micro-centrifuge tube 18. No more manual urging is required at this point to attach the layers 16a and 16b to the spindles 28 and 30, except where a new stack 16 is introduced to the system 10A.

It is also envisaged that in other embodiments of the invention, not illustrated herein, the tabs 40 and 42 are removably attachable to the spindles 28 and 30, such that the layers 16a and 16b are attached to the tabs 40 and 42 remotely from the housing 12 by a supplier (not shown) of the stack 16 and the tabs 40 and 42, such that in use the user merely mounts the stack 16 with the layers 16a and 16b already attached to the tabs 40 and 42.

Alternatively, it is also envisaged that the spindles 28 and 30 are attachably removable from the housing 12 to achieve remote attaching of the layers 16a and 16b to the tabs 40 and 42.

It is envisaged that as a third aspect of the invention, the dispenser 10 does not have a housing 12. Independently of the housing 12, the dispenser 10 becomes a dispensing apparatus (not illustrated) wherein all the components described above are located separately from each other, yet maintaining functionality by being positioned optimally by the user.

Although only certain embodiments of the invention have been described herein, it will be understood by any person skilled in the art that other modifications, variations, and possibilities of the invention are possible. Such modifications, variations and possibilities are therefore to be considered as falling within the spirit and scope of the invention and hence forming part of the invention as herein described and/or exemplified. For example, the invention can be applied in retail, the sensor 38 being configured to detect a coin of certain predetermined dimensions and the processor 36 being preset to thereby activate the motor 26 to then dispense an item. Such an item could be a stock item such as lipstick or candy.

It shall further be understood that the examples are provided for illustrating the invention further and to assist a person skilled in the art with understanding the invention and is not meant to be construed as unduly limiting the reasonable scope of the invention.

1. An automatic dispenser for dispensing sterile items from a stack, comprising:
a housing having a roller carrier arranged thereon, the roller carrier shaped and configured to rotationally support the stack;
a feed mechanism for feeding the stack towards a dispensing point, which feed mechanism is arranged in flow communication relative to the roller carrier;
a separation means extending from the feed mechanism, which separation means is shaped and configured for separating an item from the stack at the dispensing point;
an electric motor configured to drive the feed mechanism; and
a sensor arranged relative to the housing, which sensor is shaped and configured to detect a dispensing parameter, wherein in use, the dispenser is activated by a user providing the dispensing parameter to the sensor, thereby activating the feed mechanism to automatically feed the stack towards the dispensing point such that the separation means separates the item from the stack, thereby allowing for the item to be dispensed.

2. An automatic dispenser according to claim 1, further comprising an electrical power source.

3. An automatic dispenser according to claim 2 wherein the feed mechanism comprises two spaced apart wheel and cog arrangements.

4. An automatic dispenser according to claim 3 wherein the feed mechanism further comprises a third cog or a geared shaft interconnected to the motor, which third cog or geared shaft is shaped and configured to co-axially engage with the spaced apart wheel and cog arrangements.

5. An automatic dispenser according to claim 4 wherein the separation means comprises a pair of spaced apart spindles, which spindles extend from the spaced apart wheel and cog arrangement.

6. An automatic dispenser according to claim 5, further comprising a programmable computer processor arranged in electrical communication with the power source and the feed mechanism, which processor is configured to process drive the feed mechanism in a determinable manner as pre-programmed, in use.

7. An automatic dispenser according to claim 6 wherein the sensor is at least one from the set of: an electrical, electro-mechanical, mechanical and electronic arrangement, capable of detecting an electrical, electro-mechanical, mechanical or electronic parameter and being triggered by the dispensing parameter from a first state in which the arrangement does not activate the feed mechanism, to a second state in which the arrangement activate the feed mechanism.

8. A method for dispensing sterile items from a stack using an automatic dispenser comprising a housing having a roller carrier arranged thereon, the roller carrier shaped and configured to rotationally support the stack, a feed mechanism for feeding the stack towards a dispensing point, which feed mechanism is arranged in flow communication relative to the roller carrier, a separation means extending from the feed mechanism, which separation means is shaped and configured for separating an item from the stack at the dispensing point, an electric motor configured to drive the feed mechanism, and a sensor arranged relative to the housing, which sensor is shaped and configured to detect a dispensing parameter, wherein in use, the dispenser is activated by a user providing the dispensing parameter to the sensor, thereby activating the feed mechanism to automatically feed the stack towards the dispensing point such that the separation means separates the item from the stack, thereby allowing for the item to be dispensed, which method comprises the steps, of:
a. loading the stack onto the automatic dispenser;
b. securing layers of packaging of the stack onto the separation means;
c. actuating the electric motor to thereby drive the feed mechanism for feeding the stack toward a dispensing point;
d. feeding the stack via the feed mechanism toward the dispensing point located proximal to the separation means; and
e. separating an item from the stack using the separation means, which separation means is shaped and configured to separate the item from the stack.

9. A method according to claim 8 wherein step (b) precedes step (a).

10. An automatic dispensing apparatus for dispensing an item from a continuous length of packages of items, comprising:
a support shaped and configured to support the length of packages of items,
a feed mechanism for feeding the length of packages of items towards a dispensing point;
a separation means adapted to separate layers of packaging of the length of packages of items at the dispensing point;
an actuator configured to drive the feed mechanism and the separation means; and
a sensor in communication with the actuator, the sensor adapted to detect a dispensing parameter and to thereby output an initiating signal, wherein the automatic dispensing apparatus is activated upon the actuator receiving the initiating signal, thereby driving the feed mechanism to automatically feed the length of packages of items toward the dispensing point and the separation means to separate the layers of packaging at a portion of the length of packages of items proximal to the dispensing point, thereby allowing for at least the item to be dispensed.

11. An apparatus according to claim 10 wherein the support is a roller carrier adapted to rotationally support the length of packages of items as a stack.

12. An apparatus according to claim 11, comprising a power source configured to provide operational power to the apparatus in the form of direct current (DC) or alternating current (AC).

13. An apparatus according to claim 12 wherein the feed mechanism comprises a first cog and a second cog distanced apart and rotatable substantially in unison and substantially at the same velocity.

14. An apparatus according to claim 13 wherein the feed mechanism further comprises a third cog interconnected and driven by the actuator, the third cog engaged with the first and second cogs to transfer driving force from the actuator to the first and second cogs, thereby rotating co-axially therewith.

15. An apparatus according to claim 14 wherein the separation means comprises a pair of spaced apart spindles, coupled to or attachable to the first and second cogs to extend therefrom to respectively rotate in unison with the cogs.

16. An apparatus according to claim 15 wherein each spindle has a surface adapted to be attachable to a respective layer of packaging, or wherein each spindle is attached integrally to the respective layer of packaging.

17. An apparatus according to claim 16 comprising a programmable computing processor arranged in electrical communication with the sensor, the power source and the actuator, the programmable computing processor configured to control functional parameters of the actuator in a determinable manner, in use.

18. An apparatus according to claim 17 wherein the sensor is a motion sensor or a heat sensor, with the dispensing parameter respectively being movement within a predetermined distance of the sensor or heat of a predetermined magnitude.

19. An apparatus according to claim 18 wherein the actuator is a motor.

20. An automatic dispensing system comprising a continuous length of packages of items and an automatic dispensing apparatus, the automatic dispensing apparatus comprising:
a support shaped and configured to support the length of packages of items;
a feed mechanism for feeding the length of packages of items towards a dispensing point;
a separation means adapted to separate layers of packaging of the length of packages of items at the dispensing point;
an actuator configured to drive the feed mechanism and the separation means; and
a sensor in communication with the actuator, the sensor adapted to detect a dispensing parameter and to thereby output an initiating signal, wherein the apparatus is activated upon the actuator receiving the initiating signal, thereby driving the feed mechanism to automatically feed the length of packages of items towards the dispensing point and the separation means to separate the layers of packaging at a portion of the length of packages of items proximal to the dispensing point, thereby allowing for at least the item to be dispensed.
